(19) United States
(12) Patent Application Publication
(10) Pub. No.: US 2025/0160278 A1
(43) Pub. Date: May 22, 2025

(54) Brassica Oleracea Plants Resistant to Diamondback Moth

(71) Applicant: Bejo Zaden B.V., Warmenhuizen (NL)

(72) Inventors: Hubertus Theodorus Maria Janssen, Warmenhuizen (NL); Miranda Ruiter, Warmenhuizen (NL); Johannes Gerardus Maria Hoogland, Warmenhuizen (NL); Johannes Theodorus Wilhelmus Ligthart, Warmenhuizen (NL); Roelof Marinus Veenstra, Warmenhuizen (NL); Albertus Johannes Maria Schrijver, Warmenhuizen (NL)

(21) Appl. No.: 18/841,923

(22) PCT Filed: Feb. 27, 2023
(86) PCT No.: PCT/EP2023/054819

§ 371 Date: Aug. 27, 2024

(30) Foreign Application Priority Data
Feb. 28, 2022 (WO) PCT/EP2022/055009

(51) Int. Cl. A01H 6/20; A01H 1/00; A01H 1/04

(52) U.S. Cl. CPC A01H 6/203; A01H 1/045; A01H 1/125; A01H 1/127

(57) ABSTRACT

The present invention relates to Brassica oleracea plants resistant to the plant pest diamondback moth Plutella xylostella and, additionally, tolerant to the plant pathogen Xanthomonas campestris pv. campestris. The present invention further relates to methods for identifying the present plants and nucleic acid sequences allowing characterizing, or identifying, the present plants. Specifically, the present invention relates to Brassica oleracea plants resistant to the diamondback moth Plutella xylostella and the plant pathogen Xanthomonas campestris campestris, wherein said resistance to the diamondback moth Plutella xylostella and said tolerance to the plant pathogen Xanthomonas campestris campestris are obtainable from a Brassica oleracea plant deposited under deposit number NCIMB 43822.

### CROSS-REFERENCE TO RELATED APPLICATIONS

This application is the United States national phase of International Patent Application No. PCT/EP2023/054819 filed Feb. 27, 2023, and claims priority to International Patent Application No. PCT/EP2022/055009 filed Feb. 28, 2022, the disclosures of which are hereby incorporated by reference in their entireties.

### REFERENCE TO A SEQUENCE LISTING

The Sequence Listing associated with this application is filed in electronic format via Patent Center and is hereby incorporated by reference into the specification in its entirety. The name of the file containing the Sequence Listing is 2404948.xml. The size of the file is 36,802 bytes, and the file was created on Jul. 31, 2024.

### BACKGROUND OF THE INVENTION

### Field of the Invention

The present invention relates to Brassica oleracea plants resistant to the plant pest diamondback moth Plutella xylostella and, additionally, tolerant to the plant pathogen Xanthomonas campestris pv. campestris. The present invention further relates to methods for identifying the present plants and nucleic acid sequences allowing characterizing, or identifying, the present plants.

Brassica oleracea is a member of the Brassicaceae family or crucifers (Cruciferae). This species has many cultivars, which encompass several food crops. The most important of these cultivars are B. oleracea convar. capitata var. alba (white cabbage, point-headed cabbage); B. oleracea convar. capitata var. rubra (red cabbage); B. oleracea convar. botrytis var. botrytis (cauliflower, romanesco, broccoli); B. oleracea convar. botrytis var. asparagoides (sprouting broccoli); B. oleracea convar. oleracea var. gemnifera (Brussels sprouts); B. oleracea convar. capitata var. sabauda (savoy cabbage); B. oleracea convar. acephela var. sabellica (borecole); B. oleracea convar. acephela var. gongyloides (kohlrabi); and B. oleracea var. tronchuda syn. costata (Portuguese cabbage, tronchuda).

Each of these cultivars has been selected and is cultivated for a specific part of the plant. For example, white and red cabbage have prominent leaves, Brussels sprouts are formed by the axial buds of the Brassica plant, while cauliflower is white inflorescence meristem, and broccoli is formed by the flower head of the plant.

The wild Brassica oleracea plant is native to Southern and Western Europe. It is tolerant to saline conditions and is mostly restricted to coastal areas. Wild cabbage is a biennial plant. In the first year, it forms a rosette of leaves. In its second year, generally after a cold period, it produces a flower stalk of approximately 1 to 2 meters high bearing a great number of yellow flowers. From this wild species, by processes of selection and breeding, a range of different cultivars was developed over time.

The cultivar B. oleracea convar. capitata, also known as headed cabbage, has a wide phenotypic range. There are more flat varieties but also rounded and pointy-headed cabbages. Their shape can be bred according to the demand of a given market. The demand for a particular shape of the headed cabbage can change over time. Previously, markets generally preferred headed cabbage with large heads, while currently smaller cabbages are in demand.

Beyond shape, there is a rather large variation in cultivation days till harvest between varieties. Early cultivars and varieties mature in 60 days, while late types need 150 growing days. Varieties can also vary in leaf position, waxiness of the heads, the size of the head, the shape of the head, and internal core length.

Headed cabbage is consumed globally. In many countries, cabbage is a traditional food and part of many national dishes. Headed cabbage can be eaten raw, cooked, or soured. The souring process can be either due to fermentation or due to exposition to acetic acid.

Headed cabbage is a rich source of nutrients. Among the most important ones are glucosinolates and glycosides. These compounds contribute to the characteristic taste of headed cabbage. The crop further contains high amounts of vitamins B1, B6, folate, vitamin C, vitamin K and to a lesser extent the vitamins B2 and B3.

The relatively long period of cultivation makes headed cabbage more vulnerable to various diseases and disorders. Common pathogens causing disease in headed cabbage include: Xanthomonas campestris, Fusarium oxysporum, Albugo candida, Plasmodiophora brassicae, Mycosphaerella brassicae, and Plutella xylostella.

The bacterium Xanthomonas campestris pv. campestris (Xcc) is the primary cause of blackrot in Cruciferae. Blackrot is commonly found in parts of Europe, America, Africa, Asia, Australia, and Oceania. Although Brassica oleracea is economically the most important host for Xanthomonas, the bacterium can also infect other Cruciferae, weeds, and ornamental plants.

Infection with Xanthomonas campestris pv. campestris generally occurs through natural openings, such as the hydathodes of the leaves or in some cases through stomata, or wounds caused by mechanical injury. Following primary infection, the microorganism spreads through the vascular bundles. Infection initially results in a small, wilted, V-shaped infected area that extends inward from the leaf edge. The bacterium produces a sticky polysaccharide called xanthan that eventually plugs the vascular tissue inside the veins causing them to collapse and turn black. As a consequence, a part of the leaf withers, yellows and eventually dies.

Xanthomonas campestris pv. campestris is a seed transmittable disease. It can survive on seeds for years and infect a plant during an early stage of its development. Infection with the microorganism can also occur through infected plant parts, secondary host plants, and irrigation systems.

Control of the disease through chemical agents is not possible. The only measures available to combat the disease are the use of disease-free starting materials and sanitary measures, such as the removal of infected host plants. The disease-free starting material can be obtained by using pathogen-free seeds or by physically treating infected seeds. Genetically resistant plants are strongly preferred.

The diamondback moth (DBM; Plutella xylostella), sometimes called cabbage moth, is a pest of Brassica crops. DBM lays eggs on many crops belonging to the Brassicaceae family, including broccoli, cauliflower, Brussels sprouts, Chinese cabbage, kale, and kohlrabi. These plants produce glucosinolates which act as food stimulants for DBM. Other examples of affected crops are oilseed rape (Brassica napus) and canola plants (Brassica juncea). DBM can however also propagate on weeds.

This pest occurs naturally in wet, subtropical climates. Currently, DBM is present in Japan, China, Philippines, USA, Australia, remote regions, such as Reunion and New Caledonia, and colder regions, e.g. Canada. It is especially a problem in Middle and South America, including countries such as Guatemala and Mexico, as well as South Africa. Due to an increase in global temperatures as a result of climate change, Plutella xylostella has also spread north in Europe, and can now also be found in the Netherlands.

The DBM appears in May or June in the northern hemisphere. The moth is grey to brown. It is small with a wingspan of about 13 mm. There are white to silver diamond-shaped marks on the wings. The green caterpillars (up to 14 mm long) gnaw small round holes in the leaf and often leave a thin layer of leaf untouched. The caterpillars are very active and mobile. When disturbed in the crop, the moths fly upwards, and the caterpillars descend from the leaves on thin threads.

DBM is transported between plants mostly by wind. The insect can overwinter although it cannot survive cold winters and needs to re-invade colder areas in spring.

The eggs can be laid separately or in small groups on the stem or along the leaf veins, often on the underside of the leaf. When the eggs hatch, the caterpillars start to feed. In warm, dry weather an adult moth grows from an egg within 25 days. During wet, cool weather the development is slower. The insect can cause serious problems in a relatively short time, as a female can lay more than 150 eggs in 2 weeks and 2 to 5 generations per year are possible. Moreover, Brassica crops are grown in large areas worldwide and often as monocultures with year-round production. In case of infestation, DBM has plenty of feed to multiply and produce a great number of DBM individuals to invade more plants.

Infestation with DBM leads to crops being rejected as cabbage head formation is disrupted and, in the case of cauliflower and broccoli, the presence of larvae in florets renders them unsuitable for consumption. Frequently, hectares of crops are completely destroyed within a week from infestation. When no action is undertaken to combat the pest, the loss in a given area can easily be 80% and may reach 99% of the crop.

### Description of Related Art

Pesticides have been used to combat DBM, but they have various drawbacks. In the past century, the moth has developed resistance to various insecticides, e.g., pyrethroids. It can be expected that the moth will become resistant to compounds to which it is susceptible now. Another drawback of using insecticides is that these compounds are toxic to beneficial insects, specifically pollinators. Due to more strict regulations on the use of pesticides, the number of approved chemicals for use in agriculture is becoming increasingly limited. Moreover, insecticides need to be actively applied to the crop, which requires labor and is expensive, especially since the treatment needs to be repeated every 5 days. Furthermore, it is not allowed to use chemical pesticides in organic food production. There is a high demand for organic food from consumers and its market share is steadily growing, making organically grown products increasingly more relevant.

Currently, there are no commercial varieties known with absolute resistance to Plutella xylostella. However, a few commercial varieties have some resistance against Plutella xylostella. This includes the cabbage variety Izalco and related varieties, e.g., Escazu. Therefore, there is a need for Brassica plants that have a natural, genetically-encoded resistance to DBM.

Doubled haploids (DH) can be obtained by androgenesis, the development of an embryo solely from a male reproductive cell or by gynogenesis, in which the embryo genome originates exclusively from female origin. In the case of Brassica, DHs are typically plants obtained by androgenesis, derived from a single pollen grain and doubled naturally or artificially to from homozygous diploids. Doubled haploids (DH) make it possible to obtain homozygous plants in a single generation. In contrast, conventional inbreeding is slow and requires an average of six to eight generations to obtain an almost complete homozygous plant. Another disadvantage of conventional breeding is that selection in early generations is inefficient because of heterozygosity. With androgenesis, homozygosity can be achieved in one generation and more elite crosses can be evaluated and selected in a shorter time.

There are three ways in which a DH plant can be obtained: elimination of the female genome after fertilization, meiocyte-derived callogenesis, and microspore embryogenesis.

Microspore embryogenesis is a technique used for producing pure, 100% homozygous lines in a short amount of time. During microspore embryogenesis, the pollen grains or microspores are reprogrammed diverting them from their original pathway toward embryogenesis. The vacuolate microspores and young bicellular pollen are reprogrammed from the natural, gametophytic pathway toward embryogenesis. Most microspores immediately die and some continue to follow a pollen-like development. Only a few microspores are effectively induced to divide and form microspore-derived embryos as a result of the treatment.

During successful microspore embryogenesis, the microspore enlarges significantly, its nucleus moves to the cell center, and the vacuole breaks into smaller fragments. The first division of the microspore is not asymmetric but symmetric, similar to that of somatic cells. Spontaneous doubling of the haploid genome may take place at any time during culture, but it occurs most frequently during the first divisions of the embryogenic embryo. Spontaneous doubling is preferred above artificial doubling, e.g., using colchicine, but it dependents on the crop and genotype. After the first divisions, the development of the proembryo will form a complete embryo in about three to five weeks, which can be transferred to fresh media and develop into a plantlet. When the pollen parent is heterozygous, each doubled haploid plant produced is genetically different. As DH plants are 100% homozygous, microspore culture is a powerful tool to produce pure lines, which can be used as parental lines in F1-hybrids.

Molecular markers are used as a tool by researchers to introduce desired genes into cultivars. For example, the presence or absence of the genes providing resistance to plant pests such as Plutella xylostella can be detected using molecular markers. This technique saves time since laborious insect tests are not required to identify resistant plants. Testing with makers can also be performed at all stages of plant development, which is not always the case for a reliable disease test. Moreover, markers are not dependent on field circumstances, where pest pressure can vary from year to year and thus are generally less susceptible to experimental variation as compared to disease testing.

It can take considerable effort to develop informative markers. The nature of the resistance plays a role, including whether the resistance is monogenic or multigenic, dominant or recessive. The availability and quality of the insect test, the size of test populations, and the quality of the reference genome used also can affect how challenging it is to develop markers for a certain resistance.

Complex genomes can make it challenging to identify informative markers like Single Nucleotide Polymorphism (SNPs) markers. Markers used in plant breeding should be tightly linked to the loci of interest, preferably less than 5 cM genetic distance, even more preferably flanking the gene of interest and ideally being intragenic.

Due to the progress in molecular genetics, genome sequencing has become cheaper, more accessible and faster. Concomitantly, high-quality genomic data has become increasingly available and has led to the development of more informative markers, including SNP markers. For example, the AFLP or RAMP makers, which were commonly used 15 years ago, would currently not be satisfactory for contemporary plant breeding companies. As each new genetically-encoded resistance needs a tailored approach, it is currently anything but obvious to develop new markers to follow a trait.

### SUMMARY OF THE INVENTION

It is an object of the present invention, amongst other objects, to provide Brassica oleracea plants resistant to the plant pest diamondback moth Plutella xylostella and, additionally, further tolerant to the plant pathogen Xanthomonas campestris pv. campestris. It also an object of the present invention, amongst other objects, to provide means for identifying, or characterizing, the present plants. The above objects, amongst other objects, are met by the present invention as outlined in the appended claims.

Specifically, the above objects, amongst other objects, are met by the present invention by providing Brassica oleracea plants wherein the plants are resistant to the diamondback moth Plutella xylostella, wherein the plants are further tolerant to the plant pathogen Xanthomonas campestris pv. campestris, and wherein the resistance to the diamondback moth Plutella xylostella and the tolerance to the plant pathogen Xanthomonas campestris pv. campestris are obtainable from a Brassica oleracea plant deposited under deposit number NCIMB 43822 (NCIMB Limited, Craibstone Estate, 35 Ferguson Building, Bucksburn, Aberdeen AB21 9YA, United Kingdom).

The present inventors have surprisingly identified a genetic source providing resistance, or tolerance, against two major pathogens of Brassica oleracea. The present genetic source does not only provide combined resistances or tolerances, the resistances or tolerances are also stronger, or higher, than previously observed.

According to the present invention, the present Brassica oleracea plants preferably comprise in their genome:
a first genomic region located on chromosome 6, said first genomic region is characterized by one or more sequences selected from the group consisting of SEQ ID Nos. 25, 27, 29, and 31, preferably said first genomic region is comprised in a region corresponding to the region between positions 21414859 and 23635544 of the Brassica oleracea HDEM assembly; and/or
a second genomic region located on chromosome 1, said second genomic region is characterized by one or more sequences selected from the group consisting of SEQ ID Nos. 1, 3, 5, and 7, preferably said second genomic region is comprised in a region corresponding to the region between positions 11035789 and 12032392 of the Brassica oleracea HDEM assembly; and/or
a third genomic region located on chromosome 5, said third genomic region is characterized by one or more sequences selected from the group consisting of SEQ ID Nos. 17, 19, 21, and 23, preferably said third genomic region is comprised in a region corresponding to the region between positions 2137498 and 3356008 of the Brassica oleracea HDEM assembly; and/or
a fourth genomic region located on chromosome 2, said fourth genomic region is characterized by one or more sequences selected from the group consisting of SEQ ID Nos. 9, 11, 13, 15 and 33, preferably said fourth genomic region is comprised in a region corresponding to the region between positions 1654630 and 3238987 of the Brassica oleracea HDEM assembly.

The present Brassica oleracea plants as defined above are, preferably, obtained, obtainable, or are a Brassica oleracea plant deposited under deposit number NCIMB 43822. Further, the present Brassica oleracea plants are, preferably, cytoplasmic male sterile (CMS) and/or hybrids.

Considering the beneficial properties of the present Brassica oleracea plants, the present invention also relates to seeds, progeny, edible parts, egg cells, callus, suspension culture, somatic embryos, clones, embryos or plant parts of a Brassica oleracea plant as defined above.

The present invention further relates to a method for providing a Brassica oleracea as defined above, wherein the method comprises the step of introgressing, either simultaneously or separately:
a) a first genomic region as defined above; and/or
b) a second genomic region as defined above; and/or
c) a third genomic region as defined above; and/or
d) a fourth genomic region as defined above;
   into a Brassica oleracea plant not comprising the first, the second, the third and/or the fourth genomic regions, preferably, wherein the method comprises a step of providing doubled haploids, preferably by employing microspore cultures.

The present Brassica oleracea plants can be readily identified by methods for establishing the presence of a resistance providing genomic fragment, or resistance providing genomic fragments, as defined above comprising the step of determining in the genome of a Brassica oleracea plant the presence of the nucleic acid sequence or sequences selected from the group consisting of SEQ ID Nos. 1, 3, 5, 7, 9, 11, 13, 15, 17, 19, 21, 23, 25, 27, 29, 31 and 33.

In a further embodiment the present invention relates to the use of one or more of genomic sequences comprising one or more DNA sequences selected from the group consisting SEQ ID Nos. 1, 3, 5, 7, 9, 11, 13, 15, 17, 19, 21, 23, 25, 27, 29, 31 and 33 for identifying, or providing, a Brassica oleracea plant resistant to Plutella xylostella.

The present invention also relates to the use of one or more of nucleic acid sequences selected from the group consisting of SEQ ID Nos. 1, 3, 5, 7, 9, 11, 13, 15, 17, 19, 21, 23, 25, 27, 29, 31 and 33 for identifying, or providing, a Brassica oleracea plant resistant to Plutella xylostella and nucleic acid sequence selected from the group consisting of SEQ ID Nos. 1, 3, 5, 7, 9, 11, 13, 15, 17, 19, 21, 23, 25, 27, 29, 31 and 33.

### BRIEF DESCRIPTION OF THE DRAWINGS

The present invention will be further detailed in the examples below. In the examples, reference is made FIGURES wherein:

FIG. 1: shows graphs of flow cytometric data wherein the left side represents a haploid (In) plant; the middle represents a diploid or doubled haploid (2n) plant and the right side a tetraploid (4n) plant;

### EXAMPLES

### Example 1. Crossing Scheme of Plants with Plutella xylostella Resistance Including the Microspore Culture Step

### Description of the Invention

### Parent 1

TABLE 1
Breeding scheme of Parent 1 including the Microspore culture step.

| Name | | Field number | Source |
|---|---|---|---|
| Z343079*Z340129 | B2836 F1 | J5882 | Z343079*Z340129 |
| Z343079*Z340129 F1S1 | B2836 F1S1 | L12399 | J5882-1 |
| Z343079*Z340129 F1S1 | B2836 F1S1 MC | MC17600 | L12399-21 |
| MC | | | |
| Z343079*Z340129 MCS1 | B2836 MC F1S1 | T24527 | MC17600-3 |
| Z344539 | Z344539 | | |

The first cross was between Z343079 and Z340129. Z343079 is a parent line that has intermediate resistance to Xanthomonas campestris pv. campestris. Z340129 is a Bejo parent line with high resistance against Xanthomonas campestris pv. campestris. The result of this cross was B2836 F1, a Bejo variety that has intermediate resistance against Xanthomonas campestris pv. campestris.

Variety B2836 F1 was used as a donor to create a new breeding population. This was achieved by a selfing step (F1S1). The F1S1 population was used to screen for resistance against Xanthomonas campestris pv. campestris. The best individuals were selected and used for microspore culture (MC) to obtain doubled haploids (DH).

The microspore culture resulted in approximately 450 unique progenies. All of the 450 individuals were screened and 32 individuals were selected. One of these individuals became Z344539. See Example 4 for further details regarding the microspore culture.

### Parent 2

The second parent is Z344099. This is a line with intermediate resistance to Xanthomonas campestris pv. campestris.

Final step: Z344539 x Z344099->200234

The plant 200234 is a plant resistant to Plutella xylostella, which has been deposited as NCIMB 43822. Its parents Z344539 and Z344099 were chosen because of their high intermediate resistance against Xanthomonas campestris pv. campestris. In the process of developing Z344539, microspore culture was employed to generate a homozygous line with increased resistance against Xanthomonas campestris pv. campestris.

Plant 200234 was developed as a Xanthomonas resistant line. The plant was grown on a field in Guatemala, where Plutella xylostella occurs naturally and it was observed there that plant 200234 is not susceptible to this pest. Thereby, it was coincidently discovered that the line also comprises Plutella xylostella resistance.

### Example 2. Field Trial for Assessing Plutella xylostella Resistance and Plants Resistant to Plutella xylostella

To assess the level of resistance against Plutella present in the newly developed plant 200234, a full field trial was performed. The insect test was carried out under field circumstances in Guatemala at subtropical climate in the wet season. Plutella xylostella is naturally present in this area. The trial was not chemically treated. A susceptible variety was planted as border rows and in rows between the trial to increase pest pressure by allowing the multiplication and infestation of Plutella xylostella.

In the experiment trail plots of the susceptible variety, the comparator variety and the plant according to the invention were grown in lines, next to each other. The infestation occurred naturally and was not induced. The moment the crop was deemed to have matured, the pest trail was assessed.

TABLE 2
Results of the field trial for assessing Plutella xylostella
resistance (test code: XWV17GU2)

| Variety/line name | Score |
|---|---|
| Botran F1 | 3 |
| Greenboy F1 | 3 |
| Alegria F1 | 3 |
| Escazu F1 = Izalco F1 | 7 |
| 200234 | 8 |

The trial was scored using a scale ranging from 1 to 9, where 1 is fully susceptible and 9 resistant. The variety Greenboy was used as a standard of susceptible variety. Escazu and Izalco are known varieties that show tolerance for Plutella xylostella. Surprisingly, plant 200234 scored 8 and was more resistance against Plutella xylostella than Izalco, which scored 7.

### Example 3. Field Tests for Assessing Resistance Against Xanthomonas campestris pv. Campestris

An inoculum of Xanthomonas campestris pv. campestris was prepared by growing the bacterium on Yeast Dextrose Agar for 3 weeks incubated in dark at 25° C. Bacteria were scraped using water and put in a liter flask. The bacteria were suspended in water with a stirring magnet for 30 minutes. The concentration was measured by measuring absorbance with an UV-VIS spectrophotometer (PerkinElmer) and adjusted to 106 bacteria per ml. Plants were sown on 260-trays at the end of March. The plants were transplanted in the second week of May in a field in the Netherlands. Renton F1 and Morris F1 were used as susceptible control varieties.

From the second week of June on, the plants were inoculated early in the morning, when guttation takes place, for 10 consecutive weeks. Inoculation is done by spraying the bacterium suspension on the leaves using an ultra-low volume applicator. The inoculation was performed once a week. Roughly 6 weeks after the first inoculation, the first symptoms may appear. Symptoms become more apparent during cultivation. The assessment of disease resistance was carried out when the plants were ready to be harvested.

If the plant became overmature the assessment was stopped. The scale that was used ranges from 0 (completely susceptible, severe symptoms) to 9 (highly resistant, no symptoms). A score on this scale is referred to as a disease score.

TABLE 3
Results of the field test against Xanthomonas campestris
pv. campestris.

| Plant | Score |
|---|---|
| Escazu F1 | 2.6 |
| Izalco F1 | 3.8 |
| Z344539 | 6.0 |
| Z344099 | 6.0 |
| Plant 200234 | 6.6 |

### Example 4. Microspore Culture

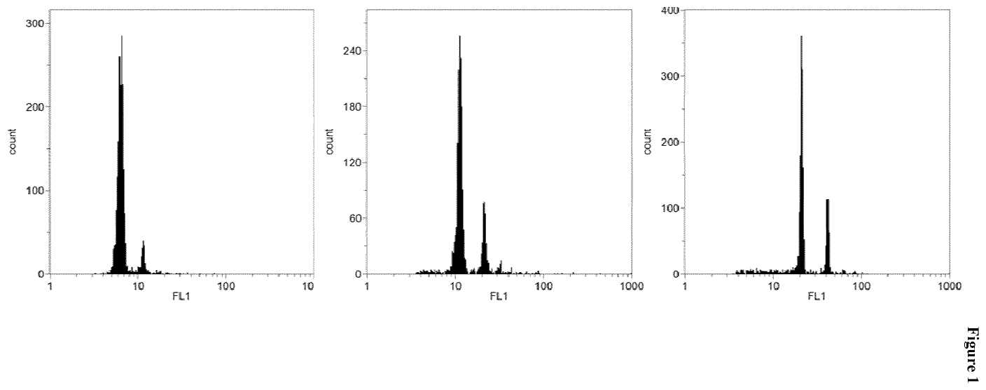

For the purpose of developing a parental line, microspore culture was used to develop doubled haploids, which are 100% homozygous lines. By employing doubled haploid plants obtained by microspore embryogenesis and using only the spontaneously doubled haploids, it was possible to greatly shorten the breeding time required to introduce the desired trait into the plants. For obtaining doubled haploid plants the procedure outlined below was followed:
Breeding lines with high quality and the desired trait were selected by the breeder. These plants were maintained and transferred to the greenhouse, see example 1 for details.
The plants were vernalized at 6° C. and grown until flowering under controlled conditions at a 16-h photoperiod at 18° C. and irrigated daily.
When flowering started, closed buds were collected and checked for the developing stage of the microspores within.
Buds that contained microspores at the late uninucleate stage, which are sensitive for reprogramming, were used for microspore isolation.
Buds were crushed in hormone free NLN media, the microspore suspension that was released, was filtered and collected in 10 ml tubes.
The suspension was washed three times by centrifugation and diluted until a concentration of 40000 msp/ml.
Microspores were given a stress treatment in the dark at 35° C. for 24 hours, to switch from the default developmental pathway toward embryogenesis.
The microspores were kept in the dark at 25° C. and after three weeks the first embryos appeared.
Microspore derived embryos at the late cotyledonary stage (0.5˜0.7 cm) were transferred to solidified media with hormones for further outgrowth and transferred to a culture room with a 16-hour photoperiod at 24° C.
After 7 weeks direct conversion of embryo to plant was determined and the ploidy level of the regenerated plantlets was checked with a Partec Flow Cytometer (FIG. 1).
Plants with a 2n level (doubled haploids) were transferred to fresh media for rooting, while haploid (In), tetraploid (4n) and mixoploid plantlets were discarded.
Rooted DH plants were transferred to the greenhouse for acclimatization and then planted out in the field.
The best performing plants were selected, selfed and crossed, and also checked for their ability as a parental line in an F1 hybrid.
One of those individuals was selected as Z344539, Parent 1 from example 1.

### Example 5. Genetic Mapping Plutella xylostella Resistance

### Population

For the genetic mapping of the Plutella xylostella (DBM) resistance, an FIS1 mapping population was made. The population is build up from the DBM resistant line A and susceptible line B. The F1 cross A*B is selfed which formed the FIS1 population. A population of 432 plants has been tested in a DBM field trial for determining the resistance levels.

### Genetic Map

For the genetic mapping informative SNP markers between line A and line B are selected. These SNP markers are developed on the DNA sequences of the respective lines A and B. The genetic map is constructed with 150 SNP markers.

### QTL Mapping

The genetic map and DBM scores are together used for the QTL mapping of the DBM resistance trait. Four QTLs with a LOD value higher than the threshold were found in the population.

In Table 4 below informative SNPs are identified for the detection of the DBM resistance QTLs. In Table 5, sequences of the SNPs from Table 4 are provided. The markers have been validated on the deposit NCIMB 43822 (Brassica oleracea 200234) and they were found to be informative.

TABLE 4
SNPs for the detection of DBM resistance in cabbage.

| | | | Physical position | Resistant | Alternative |
|---|---|---|---|---|---|
| SNP No. | KSNP | Chromosome | HDEM (bp)* | allele | allele |
| 1 | 1009-2163.1 | C1 | 11035789 | A | G |
| 2 | 1009-2161.1 | C1 | 11066385 | A | G |
| 3 | 1009-2159.1 | C1 | 11134129 | A | C |
| 4 | 1009-1500.1 | C1 | 12032392 | T | G |
| 5 | | C2 | 16546306 | T | C |
| 6 | | C2 | 17852976 | A | G |
| 7 | | C2 | 20270792 | A | G |
| 8 | | C2 | 29962502 | T | C |
| 9 | 1009-0319.1 | C2 | 32389878 | A | G |
| 10 | 1009-7049.1 | C5 | 2137498 | T | C |
| 11 | 1009-0498.1 | C5 | 2145769 | T | A |
| 12 | 1009-7050.1 | C5 | 2562211 | A | G |
| 13 | 1009-0627.1 | C5 | 3356008 | C | A |
| 14 | 1009-7060.1 | C6 | 21414859 | A | C |
| 15 | 1009-7062.1 | C6 | 22636135 | A | C |
| 16 | 1009-7063.1 | C6 | 23182069 | A | G |
| 17 | 1009-7065.1 | C6 | 23635544 | C | T |

*The Brassica oleracea HDEM assembly was used according to Belser et al,, Nature Plants Vol. 4 (2018): 879-887.

TABLE 5
Sequences of the SNPs used to detect the DBM resistance in cabbage.

| | | | Physical | |
|---|---|---|---|---|
| Seq ID | | | position in | Sequence |
| No. | SNP | Chromosome | HDEM (bp) | (SNP nucleotide is bold and in brackets) |
| 1 | 1 | C1 | 11035789 | AGGTATGTCKTCCTCCATATTCACCGATCC |
| | | | | AGCTAGATTCTGGCTGTGTYCATATGGAGA |
| | | | | CGTGTAAGAGTCTTCTKCTCCAGTTTCATC |
| | | | | ATCGGTTGTC[A]TGTCCGTAGCTGTTTGCA |
| | | | | TTTGTCTAGGAGTCTCGTCTTCCTCGTAAG |
| | | | | ACTTTGTATATGAAGTTTCTCTGCTYGTGG |
| | | | | ATTCTGAACACGTTCTCTGCAA |
| 2 | 1 | C1 | 11035789 | AGGTATGTCKTCCTCCATATTCACCGATCC |
| | | | | AGCTAGATTCTGGCTGTGTYCATATGGAGA |
| | | | | CGTGTAAGAGTCTTCTKCTCCAGTTTCATC |
| | | | | ATCGGTTGTC[G]TGTCCGTAGCTGTTTGCA |
| | | | | TTTGTCTAGGAGTCTCGTCTTCCTCGTAAG |
| | | | | ACTTTGTATATGAAGTTTCTCTGCTYGTGG |
| | | | | ATTCTGAACACGTTCTCTGCAA |
| 3 | 2 | C1 | 11066385 | ATAACATGAAATTCTAAGAGAATTGTAAAC |
| | | | | CATGTGATAATCTTTTTACAAATTGATCAG |
| | | | | [A]AATAAGAAAAAAGAGAGGTGGAATATC |
| | | | | TTCTAYATCTCTATACTCTTTCTTTCCCTT |
| | | | | GTG |
| 4 | 2 | C1 | 11066385 | ATAACATGAAATTCTAAGAGAATTGTAAAC |
| | | | | CATGTGATAATCTTTTTACAAATTGATCAG |
| | | | | [G]AATAAGAAAAAAGAGAGGTGGAATATC |
| | | | | TTCTAYATCTCTATACTCTTTCTTTCCCTT |
| | | | | GTG |
| 5 | 3 | C1 | 11134129 | AATCAACATAAAATTCCTGTGTTTGACCAC |
| | | | | TTGCAAGGACTCTATCACAGTCTTATACAT |
| | | | | [A]TGATAAGAGAAATTCAAGTGAACATAT |
| | | | | TAAAAACCAACAATTAGTGTTTGCTCACAG |
| | | | | AAG |
| 6 | 3 | C1 | 11134129 | AATCAACATAAAATTCCTGTGTTTGACCAC |
| | | | | TTGCAAGGACTCTATCACAGTCTTATACAT |
| | | | | [C]TGATAAGAGAAATTCAAGTGAACATAT |
| | | | | TAAAAACCAACAATTAGTGTTTGCTCACAG |
| | | | | AAG |
| 7 | 4 | C1 | 12032392 | AGCGGTTTGTCTGACGCCTCTCCGTCTTCTT |
| | | | | TAATCCACGGATGTTTTAGTACTTCACTCG |
| | | | | CTGTTAGCCGGTCTTTAGGGTCATATGTCA |
| | | | | ACATTTTCTTCACAAGATCTTTGGCACCGTT |
| | | | | GGATACA[T]CAGGCCATGGATCAGCTGAA |
| | | | | AAATCAAGCTTTCCTTCTAGAATGGCATCG |
| | | | | AAGATCCCTGTCTCATTTTCTCCCCAGAAA |
| | | | | GGTGGGACACCACTGAGAAGGATGTACAG |
| | | | | AATCACACCGGCACTCCAAATATCAGCCTC |
| | | | | TGGTCCATAGTTCCGTTTTAAAACTTCCGG |
| | | | | GGCAACATAGTATGCACTTCCAACAAGATC |
| | | | | C |
| 8 | 4 | C1 | 12032392 | AGCGGTTTGTCTGACGCCTCTCCGTCTTCTT |
| | | | | TAATCCACGGATGTTTTAGTACTTCACTCG |
| | | | | CTGTTAGCCGGTCTTTAGGGTCATATGTCA |
| | | | | ACATTTTCTTCACAAGATCTTTGGCACCGTT |
| | | | | GGATACA[G]CAGGCCATGGATCAGCTGAA |
| | | | | AAATCAAGCTTTCCTTCTAGAATGGCATCG |
| | | | | AAGATCCCTGTCTCATTTTCTCCCCAGAAA |
| | | | | GGTGGGACACCACTGAGAAGGATGTACAG |
| | | | | AATCACACCGGCACTCCAAATATCAGCCTC |
| | | | | TGGTCCATAGTTCCGTTTTAAAACTTCCGG |
| | | | | GGCAACATAGTATGCACTTCCAACAAGATC |
| | | | | C |
| 9 | 5 | C2 | 16546306 | CAGCAAGCTTCAGATAAATCCATTGTGATA |
| | | | | AGTGAAATTCTGGACTCAAGAACAAAGAAC |
| | | | | [T]TTGTCTCTGTCTCCAGAATAAGTGATT |
| | | | | ACGTACTATCCAACGAGCTAGTTAGTATGG |
| | | | | CTC |
| 10 | 5 | C2 | 16546306 | CAGCAAGCTTCAGATAAATCCATTGTGATA |
| | | | | AGTGAAATTCTGGACTCAAGAACAAAGAAC |
| | | | | [C]TTGTCTCTGTCTCCAGAATAAGTGATT |
| | | | | ACGTACTATCCAACGAGCTAGTTAGTATGG |
| | | | | CTC |
| 11 | 6 | C2 | 17852976 | GAGACACTCTATCCTTATACATTGCCATCA |
| | | | | CCTCCTCGGCTGTTAGCAGCGTCCTAATAC |
| | | | | [A]AGAAGACCGTGGTGAACAGAAACATAT |
| | | | | TTTCTACACCAGCAAGCGGATGATGGAACC |
| | | | | GGA |
| 12 | 6 | C2 | 17852976 | GAGACACTCTATCCTTATACATTGCCATCA |
| | | | | CCTCCTCGGCTGTTAGCAGCGTCCTAATAC |
| | | | | [G]AGAAGACCGTGGTGAACAGAAACATAT |
| | | | | TTTCTACACCAGCAAGCGGATGATGGAACC |
| | | | | GGA |
| 13 | 7 | C2 | 20270792 | TTGTTCTCCGCTTTCTTCGCAAATTAGCACT |
| | | | | CCGCTTACTGCGTGTTTGGAGGTTGCCAC[A] |
| | | | | TACAAAAAGAGGGTCTCTCCTATGATTGGT |
| | | | | TTGACAGAATGGGCGGCTCGCTGATATATG |
| 14 | 7 | C2 | 20270792 | TTGTTCTCCGCTTTCTTCGCAAATTAGCACT |
| | | | | CCGCTTACTGCGTGTTTGGAGGTTGCCAC[G] |
| | | | | TACAAAAAGAGGGTCTCTCCTATGATTGGT |
| | | | | TTGACAGAATGGGCGGCTCGCTGATATATG |
| 15 | 8 | C2 | 29962502 | GATAAAATAAAATTGAATTGTTTTCAACAT |
| | | | | AGATGAGTGAAAGCAGTGAATCATGATATT |
| | | | | [T]TTTGGTCTAGGTTTTGACAACATATGT |
| | | | | TATAGTATTGTATGTATTCTTAGGGTTAGA |
| | | | | TTT |
| 16 | 8 | C2 | 29962502 | GATAAAATAAAATTGAATTGTTTTCAACAT |
| | | | | AGATGAGTGAAAGCAGTGAATCATGATAT |
| | | | | T[C] |
| | | | | TTTGGTCTAGGTTTTGACAACATATGTTAT |
| | | | | AGTATTGTATGTATTCTTAGGGTTAGATTT |
| 33 | 9 | C2 | 32389878 | GGCAACTTGTGGCGCTGTACTTCTTGTCTCT |
| | | | | TTGGTTTCGTCTTTGGGAATGGCCTTTTT[A] |
| | | | | TCAAAGGCAGCTAAAGCTTGCTGAAAACA |
| | | | | TAACATTCTCTATCTGTTAGACTTTCATTTA |
| 34 | 9 | C2 | 32389878 | GGCAACTTGTGGCGCTGTACTTCTTGTCTCT |
| | | | | TTGGTTTCGTCTTTGGGAATGGCCTTTTT[G] |
| | | | | TCAATGGCAGCTAAAGCTTGCTGAAAACAT |
| | | | | AACATTCTCTATCTGTTAGACTTTCATTTA |
| 17 | 10 | C5 | 2137498 | CTAAATCCTGTGTGGATTTGGCTGGCAGCT |
| | | | | TTTGTCTAAAATAAAATCTAGATAACCACA |
| | | | | ACATTCTTCAACGCTGTTACTCTGCCCTCAT |
| | | | | TATGTATGG[T]ATGCCTATGATATTGCGCT |
| | | | | TGTCTTGTCTTGATGACGTTTTCATTTGTTT |
| | | | | TTTCTGCTTCAGATGAAGTGGTGTCCTGCC |
| | | | | CCAGGATGTGAGAATGCAAT |
| 18 | 10 | C5 | 2137498 | CTAAATCCTGTGTGGATTTGGCTGGCAGCT |
| | | | | TTTGTCTAAAATAAAATCTAGATAACCACA |
| | | | | ACATTCTTCAACGCTGTTACTCTGCCCTCAT |
| | | | | TATGTATGG[C]ATGCCTATGATATTGCGCT |
| | | | | TGTCTTGTCTTGATGACGTTTTCATTTGTTT |
| | | | | TTTCTGCTTCAGATGAAGTGGTGTCCTGCC |
| | | | | CCAGGATGTGAGAATGCAAT |
| 19 | 11 | C5 | 2145769 | TCTTGCAAGATGTTGATCTCATTGTGAAAA |
| | | | | ATGCCAAGGCCTACAATGGAGATGATTAC |
| | | | | GCTGGAGCGAGAATTGTCAGTAGAGCCTA |
| | | | | CGAGCTTCGGGATGTAGTGCATGGGATGCT |
| | | | | GGCGCAGATGGACCCAGCACTGCTAACAT |
| | | | | ATTGTGACAAAATCGC[T]GCTGAAGGTGGT |
| | | | | CCTTCACAGATACCAGATGATCTGAGTGAA |
| | | | | TCTATCCTTGGTTTAGCTCCTGTGGTGCAG |
| | | | | ATGGGGACTGTTACTAGAGCAAGTGCCCG |
| | | | | GCTTCGAAACATGCAGCCAGAGGTTAATAT |
| | | | | AGATCGAGATCATGAAGGTTTTAAAAAGC |
| | | | | CTAAGAAAACAGCTGATGCTGCCTGTACAG |
| | | | | ATTCAGCTGC |
| 20 | 11 | C5 | 2145769 | TCTTGCAAGATGTTGATCTCATTGTGAAAA |
| | | | | ATGCCAAGGCCTACAATGGAGATGATTAC |
| | | | | GCTGGAGCGAGAATTGTCAGTAGAGCCTA |
| | | | | CGAGCTTCGGGATGTAGTGCATGGGATGCT |
| | | | | GGCGCAGATGGACCCAGCACTGCTAACAT |
| | | | | ATTGTGACAAAATCGC[A]GCTGAAGGTGGT |
| | | | | CCTTCACAGATACCAGATGATCTGAGTGAA |
| | | | | TCTATCCTTGGTTTAGCTCCTGTGGTGCAG |
| | | | | ATGGGGACTGTTACTAGAGCAAGTGCCCG |
| | | | | GCTTCGAAACATGCAGCCAGAGGTTAATAT |
| | | | | AGATCGAGATCATGAAGGTTTTAAAAAGC |
| | | | | CTAAGAAAACAGCTGATGCTGCCTGTACAG |
| | | | | ATTCAGCTGC |
| 21 | 12 | C5 | 2562211 | AACAACACACGGTATAAATGAAAATACCT |
| | | | | GAGTGTGAAGATGTCAACGACGCAGCTGA |
| | | | | ATCCCTTAGTCTCCTTACACTCTCAGCAGC |
| | | | | ATCCAAAGTTTC[A]GAATACTCCTCTGATG |
| | | | | TTGGTTCAATCAGACTATCCCTCTTAAGGC |
| | | | | CCAGCAGCGCATTTTCGGAGGCCTAGGTCA |
| | | | | ACGTCAGAACAATGTCATATACCG |
| 22 | 12 | C5 | 2562211 | AACAACACACGGTATAAATGAAAATACCT |
| | | | | GAGTGTGAAGATGTCAACGACGCAGCTGA |
| | | | | ATCCCTTAGTCTCCTTACACTCTCAGCAGC |
| | | | | ATCCAAAGTTTC[G]GAATACTCCTCTGATG |
| | | | | TTGGTTCAATCAGACTATCCCTCTTAAGGC |
| | | | | CCAGCAGCGCATTTTCGGAGGCCTAGGTCA |
| | | | | ACGTCAGAACAATGTCATATACCG |
| 23 | 13 | C5 | 3356008 | ACAGGAACAGCGTTTTGCTCAACCATCTGT |
| | | | | TTCTGTCTGCCGCGAACAACCTCTGGCTGC |
| | | | | CAAACGTTCAAAGCTACTGATGCTTGCCTG |
| | | | | ACTCTCTGCAGATTTGAGAACTCAACAGAA |
| | | | | GCAGCCTGCAACCTCTCTGCCTCCTCWGAT |
| | | | | AACCTCCTCTCCAATTCACGAATCCTCCTG |
| | | | | CCCCACTCCGAAATCTCGCT[C]TCCTTCC |
| | | | | TCAGCAGAGATCTTCTATGAGCAGCTAACC |
| | | | | TCCTCTCCTTCTCCCTCTCAAACCTTGTCC |
| | | | | TGTAGATAAACTTGGTCGGAGATTCAGAAG |
| | | | | AGGAAGAAGAAGAAGTAGAGTCGGTGTGGG |
| | | | | TGGGGAAATCAATGGCGAGCAGAAAGTTCC |
| | | | | AGAGGCAATCTTCGGAGGATAGTCGGCAG |
| | | | | AGACGGCGAGAGGA |
| 24 | 13 | C5 | 3356008 | ACAGGAACAGCGTTTTGCTCAACCATCTGT |
| | | | | TTCTGTCTGCCGCGAACAACCTCTGGCTGC |
| | | | | CAAACGTTCAAAGCTACTGATGCTTGCCTG |
| | | | | ACTCTCTGCAGATTTGAGAACTCAACAGAA |
| | | | | GCAGCCTGCAACCTCTCTGCCTCCTCWGAT |
| | | | | AACCTCCTCTCCAATTCACGAATCCTCCTG |
| | | | | CCCCACTCCGAAATCTCGCT[A]TCCTTCC |
| | | | | TCAGCAGAGATCTTCTATGAGCAGCTAACC |
| | | | | TCCTCTCCTTCTCCCTCTCAAACCTTGTCC |
| | | | | TGTAGATAAACTTGGTCGGAGATTCAGAAG |
| | | | | AGGAAGAAGAAGAAGTAGAGTCGGTGTGGG |
| | | | | TGGGGAAATCAATGGCGAGCAGAAAGTTCC |
| | | | | AGAGGCAATCTTCGGAGGATAGTCGGCAG |
| | | | | AGACGGCGAGAGGA |
| 25 | 14 | C6 | 21414859 | CTGTTACTTAGAAACTTCATTTTTTTTTATA |
| | | | | GGTTTTTGCTAACTGTTAGATACAAAGTATT |
| | | | | ATTATTTGAAGTTTTTTTCATTGTTGTCAAA |
| | | | | TCTCGTT[A]TCTTCAGTTACAAAGCATTCT |
| | | | | CCAATAAATCTCTTTGTCTCTTTTTTACCCA |
| | | | | AAAAACAAAATCTATCTGTCTTTGTTGTTA |
| | | | | AATTTTTCATGGGGTCTT |
| 26 | 14 | C6 | 21414859 | CTGTTACTTAGAAACTTCATTTTTTTTTATA |
| | | | | GGTTTTTGCTAACTGTTAGATACAAAGTAT |
| | | | | TATTATTTGAAGTTTTTTTCATTGTTGTCAA |
| | | | | ATCTCGTT[C]TCTTCAGTTACAAAGCATTCT |
| | | | | CCAATAAATCTCTTTGTCTCTTTTTTACCCA |
| | | | | AAAAACAAAATCTATCTGTCTTTGTTGTTA |
| | | | | AATTTTTCATGGGGTCTT |
| 27 | 15 | C6 | 22636135 | ACTGGCTGAGTGTCCTCTATTATTTCCTCTA |
| | | | | CAGGCTGAATGTCACCATGCTTCTCTTCCA |
| | | | | CCACCTGAGCAACTTGCTCAACTGGTTCAG |
| | | | | TGTTATCCA[A]CATTTCCTCAATCAGTTGTG |
| | | | | TCTCATCTCCTACTTCCGTAACAAGAATGT |
| | | | | CATCGTCAGCATGCTTCTCCTCCACCAATT |
| | | | | GTGCCTCCTGAGCTACTTCT |
| 28 | 15 | C6 | 22636135 | ACTGGCTGAGTGTCCTCTATTATTTCCTCTA |
| | | | | CAGGCTGAATGTCACCATGCTTCTCTTCCA |
| | | | | CCACCTGAGCAACTTGCTCAACTGGTTCAG |
| | | | | TGTTATCCA[C]CATTTCCTCAATCAGTTGTG |
| | | | | TCTCATCTCCTACTTCCGTAACAAGAATGT |
| | | | | CATCGTCAGCATGCTTCTCCTCCACCAATT |
| | | | | GTGCCTCCTGAGCTACTTCT |
| 29 | 16 | C6 | 23182069 | TTATTAGATATGAGGAAGGAGAATGTCAA |
| | | | | ACCTAGCATAAGAGGAATTCTGGAGAGAT |
| | | | | AGTAGAACCAGATTAAGCTTACAAAATATT |
| | | | | TGTATGGTTAAT[A]AGTTTTGAAGTTCAAA |
| | | | | ATCACAATTCAGTGGTCACTACAAGAAAAC |
| | | | | ACATGGTTAACGACGAAAATTAACGAGGA |
| | | | | AAAACAATCCTCGTAAATTTGCGTC |
| 30 | 16 | C6 | 23182069 | TTATTAGATATGAGGAAGGAGAATGTCAA |
| | | | | ACCTAGCATAAGAGGAATTCTGGAGAGAT |
| | | | | AGTAGAACCAGATTAAGCTTACAAAATATT |
| | | | | TGTATGGTTAAT[G]AGTTTTGAAGTTCAAA |
| | | | | ATCACAATTCAGTGGTCACTACAAGAAAAC |
| | | | | ACATGGTTAACGACGAAAATTAACGAGGA |
| | | | | AAAACAATCCTCGTAAATTTGCGTC |
| 31 | 17 | C6 | 23635544 | TCCATGCTTCCTTCATCAGTTCCTGGGTAA |
| | | | | ACAAGGATTTTACCATGGCCAGCAATTCCC |
| | | | | GGTCCAGCTCTAACCGCAATCGGTTTACCC |
| | | | | CAACCAAAAT[C]GTTTCCAAACACATTGAA |
| | | | | CCGTGGAGAGCTAGCAACAACGAGCGAAT |
| | | | | TATTCACCATCAAACCATTCGGTATTTTAG |
| | | | | GGTTCTTCACCCAATTTTCAGCA |
| 32 | 17 | C6 | 23635544 | TCCATGCTTCCTTCATCAGTTCCTGGGTAA |
| | | | | ACAAGGATTTTACCATGGCCAGCAATTCCC |
| | | | | GGTCCAGCTCTAACCGCAATCGGTTTACCC |
| | | | | CAACCAAAAT[T]GTTTCCAAACACATTGAA |
| | | | | CCGTGGAGAGCTAGCAACAACGAGCGAAT |
| | | | | TATTCACCATCAAACCATTCGGTATTTTAG |
| | | | | GGTTCTTCACCCAATTTTCAGCA |

The abbreviations are according to IUPAC nucleotide code:

| Symbol | Nucleotide Base |
|---|---|
| A | Adenine |
| C | Cytosine |
| G | Guanine |
| T | Thymine |
| N | A or C or G or T |
| M | A or C |
| R | A or G |
| W | A or T |
| S | C or G |
| Y | C or T |
| K | G or T |
| V | Not T |
| H | Not G |
| D | Not C |
| B | Not A |

Information about the Deposit NCIMB 43822.

A deposit of a plant according to the invention, Brassica oleracea 200234, deposit NCIMB 43822 was deposited at NCIMB Limited, Craibetone Estate, 35 Ferguson Building, Bucksburn, Aberdeen AB21 9YA, United Kingdom on Jul. 26, 2021.

1. Brassica oleracea plant wherein said plant is resistant to the diamondback moth Plutella xylostella, wherein said plant is further tolerant to the plant pathogen Xanthomonas campestris pv. campestris, and wherein said resistance to the diamondback moth Plutella xylostella and said tolerance to the plant pathogen Xanthomonas campestris pv. campestris are obtainable from a Brassica oleracea plant deposited under deposit number NCIMB 43822.

2. The Brassica oleracea plant according to claim 1, wherein said plant further comprises in its genome:
a first genomic region located on chromosome 6, said first genomic region is characterized by one or more sequences selected from the group consisting of SEQ ID Nos. 25, 27, 29, and 31.

3. The Brassica oleracea plant according to claim 1, wherein said plant comprises in its genome:
a second genomic region located on chromosome 1, said second genomic region is characterized by one or more sequences selected from the group consisting of SEQ ID Nos. 1, 3, 5, and 7.

4. The Brassica oleracea plant according to claim 1, wherein said plant further comprises in its genome:
a third genomic region located on chromosome 5, said third genomic region is characterized by one or more sequences selected from the group consisting of SEQ ID Nos. 17, 19, 21, and 23.

5. Brassica oleracea plant according to claim 1, wherein said plant further comprises in its genome:
a fourth genomic region located on chromosome 2, said fourth genomic region is characterized by one or more sequences selected from the group consisting of SEQ ID Nos. 9, 11, 13, 15, and 33.

6. (canceled)

7. The Brassica oleracea plant according to claim 1, wherein said plant is obtained, obtainable, or is a Brassica oleracea plant deposited under deposit number NCIMB 43822.

8. The Brassica oleracea plant according to claim 1, wherein said plant is cytoplasmic male sterile (CMS).

9. The Brassica oleracea plant according to claim 1, wherein said plant is a hybrid plant.

10. Seeds, progeny, edible parts, egg cells, callus, suspension culture, somatic embryos, clones, embryos, or plant parts of a Brassica oleracea plant according to claim 1.

11. A method for providing a Brassica oleracea according to claim 1, wherein the method comprises the step of introgressing, either simultaneously or separately:
a) one or more sequences selected from the group consisting of SEQ ID Nos. 25, 27, 29, and 31; and/or
b) one or more sequences selected from the group consisting of SEQ ID Nos. 1, 3, 5, and 7, and/or
c) one or more sequences selected from the group consisting of SEQ ID Nos. 17, 19, 21, and 23, and/or
d) one or more sequences selected from the group consisting of SEQ ID Nos. 9, 11, 13, 15, and 33,
into a Brassica oleracea plant not comprising the one or more sequences.

12. A method for identifying a Brassica oleracea plant being resistant to Plutella xylostella, the method comprises the step of establishing the presence of a resistance providing genomic region, or resistance providing genomic regions, by determining in the genome of said plant the presence of one or more of the corresponding nucleic acid sequences selected from the group consisting of SEQ ID Nos. 1, 3, 5, 7, 9, 11, 13, 15, 17, 19, 21, 23, 25, 27, 29, 31 and 33.

13. (canceled)

14. (canceled)

15. The Brassica oleracea plant according to claim 1, wherein said plant is not exclusively obtainable by an essentially biological process.

16. The Brassica oleracea plant according to claim 2, wherein said first genomic region is between positions 21414859 and 23635544 of the Brassica oleracea HDEM assembly.

17. The Brassica oleracea plant according to claim 3, wherein said second genomic region is between positions 11035789 and 12032392 of the Brassica oleracea HDEM assembly.

18. The Brassica oleracea plant according to claim 4, wherein said third genomic region is between positions 2137498 and 3356008 of the Brassica oleracea HDEM assembly.

19. The Brassica oleracea plant according to claim 5, wherein said fourth genomic region is between positions 1654630 and 3238987 of the Brassica oleracea HDEM assembly.


SEQUENCE LISTING (34 sequences)

SEQ ID NO: 1 - DNA, 201 bp, Brassica oleracea
  source 1..201: mol_type = genomic DNA
aggtatgtcktcctccatattcaccgatccagctagattctggctgtgtycatatggagacgtgtaagagtcttctkctccagtttcatcatcggttgtcatgtccgtagctgtttgcatttgtctaggagtctcgtcttcctcgtaagactttgtatatgaagtttctctgctygtggattctgaacacgttctctgcaa

SEQ ID NO: 2 - DNA, 201 bp, Brassica oleracea
  source 1..201: mol_type = genomic DNA
aggtatgtcktcctccatattcaccgatccagctagattctggctgtgtycatatggagacgtgtaagagtcttctkctccagtttcatcatcggttgtcgtgtccgtagctgtttgcatttgtctaggagtctcgtcttcctcgtaagactttgtatatgaagtttctctgctygtggattctgaacacgttctctgcaa

SEQ ID NO: 3 - DNA, 121 bp, Brassica oleracea
  source 1..121: mol_type = genomic DNA
ataacatgaaattctaagagaattgtaaaccatgtgataatctttttacaaattgatcagaaataagaaaaaagagaggtggaatatcttctayatctctatactctttctttcccttgtg

SEQ ID NO: 4 - DNA, 121 bp, Brassica oleracea
  source 1..121: mol_type = genomic DNA
ataacatgaaattctaagagaattgtaaaccatgtgataatctttttacaaattgatcaggaataagaaaaaagagaggtggaatatcttctayatctctatactctttctttcccttgtg

SEQ ID NO: 5 - DNA, 121 bp, Brassica oleracea
  source 1..121: mol_type = genomic DNA
aatcaacataaaattcctgtgtttgaccacttgcaaggactctatcacagtcttatacatatgataagagaaattcaagtgaacatattaaaaaccaacaattagtgtttgctcacagaag

SEQ ID NO: 6 - DNA, 121 bp, Brassica oleracea
  source 1..121: mol_type = genomic DNA
aatcaacataaaattcctgtgtttgaccacttgcaaggactctatcacagtcttatacatctgataagagaaattcaagtgaacatattaaaaaccaacaattagtgtttgctcacagaag

SEQ ID NO: 7 - DNA, 330 bp, Brassica oleracea
  source 1..330: mol_type = genomic DNA
agcggtttgtctgacgcctctccgtcttctttaatccacggatgttttagtacttcactcgctgttagccggtctttagggtcatatgtcaacattttcttcacaagatctttggcaccgttggatacatcaggccatggatcagctgaaaaatcaagctttccttctagaatggcatcgaagatccctgtctcattttctccccagaaaggtgggacaccactgagaaggatgtacagaatcacaccggcactccaaatatcagcctctggtccatagttccgttttaaaacttccggggcaacatagtatgcacttccaacaagatcc

SEQ ID NO: 8 - DNA, 330 bp, Brassica oleracea
  source 1..330: mol_type = genomic DNA
agcggtttgtctgacgcctctccgtcttctttaatccacggatgttttagtacttcactcgctgttagccggtctttagggtcatatgtcaacattttcttcacaagatctttggcaccgttggatacagcaggccatggatcagctgaaaaatcaagctttccttctagaatggcatcgaagatccctgtctcattttctccccagaaaggtgggacaccactgagaaggatgtacagaatcacaccggcactccaaatatcagcctctggtccatagttccgttttaaaacttccggggcaacatagtatgcacttccaacaagatcc

SEQ ID NO: 9 - DNA, 121 bp, Brassica oleracea
  source 1..121: mol_type = genomic DNA
cagcaagcttcagataaatccattgtgataagtgaaattctggactcaagaacaaagaactttgtctctgtctccagaataagtgattacgtactatccaacgagctagttagtatggctc

SEQ ID NO: 10 - DNA, 121 bp, Brassica oleracea
  source 1..121: mol_type = genomic DNA
cagcaagcttcagataaatccattgtgataagtgaaattctggactcaagaacaaagaaccttgtctctgtctccagaataagtgattacgtactatccaacgagctagttagtatggctc

SEQ ID NO: 11 - DNA, 121 bp, Brassica oleracea
  source 1..121: mol_type = genomic DNA
gagacactctatccttatacattgccatcacctcctcggctgttagcagcgtcctaatacaagaagaccgtggtgaacagaaacatattttctacaccagcaagcggatgatggaaccgga

SEQ ID NO: 12 - DNA, 121 bp, Brassica oleracea
  source 1..121: mol_type = genomic DNA
gagacactctatccttatacattgccatcacctcctcggctgttagcagcgtcctaatacgagaagaccgtggtgaacagaaacatattttctacaccagcaagcggatgatggaaccgga

SEQ ID NO: 13 - DNA, 121 bp, Brassica oleracea
  source 1..121: mol_type = genomic DNA
ttgttctccgctttcttcgcaaattagcactccgcttactgcgtgtttggaggttgccacatacaaaaagagggtctctcctatgattggtttgacagaatgggcggctcgctgatatatg

SEQ ID NO: 14 - DNA, 121 bp, Brassica oleracea
  source 1..121: mol_type = genomic DNA
ttgttctccgctttcttcgcaaattagcactccgcttactgcgtgtttggaggttgccacgtacaaaaagagggtctctcctatgattggtttgacagaatgggcggctcgctgatatatg

SEQ ID NO: 15 - DNA, 121 bp, Brassica oleracea
  source 1..121: mol_type = genomic DNA
gataaaataaaattgaattgttttcaacatagatgagtgaaagcagtgaatcatgatattttttggtctaggttttgacaacatatgttatagtattgtatgtattcttagggttagattt

SEQ ID NO: 16 - DNA, 121 bp, Brassica oleracea
  source 1..121: mol_type = genomic DNA
gataaaataaaattgaattgttttcaacatagatgagtgaaagcagtgaatcatgatattctttggtctaggttttgacaacatatgttatagtattgtatgtattcttagggttagattt

SEQ ID NO: 17 - DNA, 201 bp, Brassica oleracea
  source 1..201: mol_type = genomic DNA
ctaaatcctgtgtggatttggctggcagcttttgtctaaaataaaatctagataaccacaacattcttcaacgctgttactctgccctcattatgtatggtatgcctatgatattgcgcttgtcttgtcttgatgacgttttcatttgttttttctgcttcagatgaagtggtgtcctgccccaggatgtgagaatgcaat

SEQ ID NO: 18 - DNA, 201 bp, Brassica oleracea
  source 1..201: mol_type = genomic DNA
ctaaatcctgtgtggatttggctggcagcttttgtctaaaataaaatctagataaccacaacattcttcaacgctgttactctgccctcattatgtatggcatgcctatgatattgcgcttgtcttgtcttgatgacgttttcatttgttttttctgcttcagatgaagtggtgtcctgccccaggatgtgagaatgcaat

SEQ ID NO: 19 - DNA, 364 bp, Brassica oleracea
  source 1..364: mol_type = genomic DNA
tcttgcaagatgttgatctcattgtgaaaaatgccaaggcctacaatggagatgattacgctggagcgagaattgtcagtagagcctacgagcttcgggatgtagtgcatgggatgctggcgcagatggacccagcactgctaacatattgtgacaaaatcgctgctgaaggtggtccttcacagataccagatgatctgagtgaatctatccttggtttagctcctgtggtgcagatggggactgttactagagcaagtgcccggcttcgaaacatgcagccagaggttaatatagatcgagatcatgaaggttttaaaaagcctaagaaaacagctgatgctgcctgtacagattcagctgc

SEQ ID NO: 20 - DNA, 364 bp, Brassica oleracea
  source 1..364: mol_type = genomic DNA
tcttgcaagatgttgatctcattgtgaaaaatgccaaggcctacaatggagatgattacgctggagcgagaattgtcagtagagcctacgagcttcgggatgtagtgcatgggatgctggcgcagatggacccagcactgctaacatattgtgacaaaatcgcagctgaaggtggtccttcacagataccagatgatctgagtgaatctatccttggtttagctcctgtggtgcagatggggactgttactagagcaagtgcccggcttcgaaacatgcagccagaggttaatatagatcgagatcatgaaggttttaaaaagcctaagaaaacagctgatgctgcctgtacagattcagctgc

SEQ ID NO: 21 - DNA, 201 bp, Brassica oleracea
  source 1..201: mol_type = genomic DNA
aacaacacacggtataaatgaaaatacctgagtgtgaagatgtcaacgacgcagctgaatcccttagtctccttacactctcagcagcatccaaagtttcagaatactcctctgatgttggttcaatcagactatccctcttaaggcccagcagcgcattttcggaggcctaggtcaacgtcagaacaatgtcatataccg

SEQ ID NO: 22 - DNA, 201 bp, Brassica oleracea
  source 1..201: mol_type = genomic DNA
aacaacacacggtataaatgaaaatacctgagtgtgaagatgtcaacgacgcagctgaatcccttagtctccttacactctcagcagcatccaaagtttcggaatactcctctgatgttggttcaatcagactatccctcttaaggcccagcagcgcattttcggaggcctaggtcaacgtcagaacaatgtcatataccg

SEQ ID NO: 23 - DNA, 401 bp, Brassica oleracea
  source 1..401: mol_type = genomic DNA
acaggaacagcgttttgctcaaccatctgtttctgtctgccgcgaacaacctctggctgccaaacgttcaaagctactgatgcttgcctgactctctgcagatttgagaactcaacagaagcagcctgcaacctctctgcctcctcwgataacctcctctccaattcacgaatcctcctgccccactccgaaatctcgctctccttcctcagcagagatcttctatgagcagctaacctcctctccttctccctctcaaaccttgtcctgtagataaacttggtcggagattcagaagaggaagaagaagaagtagagtcggtgtgggtggggaaatcaatggcgagcagaaagttccagaggcaatcttcggaggatagtcggcagagacggcgagagga

SEQ ID NO: 24 - DNA, 401 bp, Brassica oleracea
  source 1..401: mol_type = genomic DNA
acaggaacagcgttttgctcaaccatctgtttctgtctgccgcgaacaacctctggctgccaaacgttcaaagctactgatgcttgcctgactctctgcagatttgagaactcaacagaagcagcctgcaacctctctgcctcctcwgataacctcctctccaattcacgaatcctcctgccccactccgaaatctcgctatccttcctcagcagagatcttctatgagcagctaacctcctctccttctccctctcaaaccttgtcctgtagataaacttggtcggagattcagaagaggaagaagaagaagtagagtcggtgtgggtggggaaatcaatggcgagcagaaagttccagaggcaatcttcggaggatagtcggcagagacggcgagagga

SEQ ID NO: 25 - DNA, 201 bp, Brassica oleracea
  source 1..201: mol_type = genomic DNA
ctgttacttagaaacttcatttttttttataggtttttgctaactgttagatacaaagtattattatttgaagtttttttcattgttgtcaaatctcgttatcttcagttacaaagcattctccaataaatctctttgtctcttttttacccaaaaaacaaaatctatctgtctttgttgttaaatttttcatggggtctt

SEQ ID NO: 26 - DNA, 201 bp, Brassica oleracea
  source 1..201: mol_type = genomic DNA
ctgttacttagaaacttcatttttttttataggtttttgctaactgttagatacaaagtattattatttgaagtttttttcattgttgtcaaatctcgttctcttcagttacaaagcattctccaataaatctctttgtctcttttttacccaaaaaacaaaatctatctgtctttgttgttaaatttttcatggggtctt

SEQ ID NO: 27 - DNA, 201 bp, Brassica oleracea
  source 1..201: mol_type = genomic DNA
actggctgagtgtcctctattatttcctctacaggctgaatgtcaccatgcttctcttccaccacctgagcaacttgctcaactggttcagtgttatccaacatttcctcaatcagttgtgtctcatctcctacttccgtaacaagaatgtcatcgtcagcatgcttctcctccaccaattgtgcctcctgagctacttct

SEQ ID NO: 28 - DNA, 201 bp, Brassica oleracea
  source 1..201: mol_type = genomic DNA
actggctgagtgtcctctattatttcctctacaggctgaatgtcaccatgcttctcttccaccacctgagcaacttgctcaactggttcagtgttatccaccatttcctcaatcagttgtgtctcatctcctacttccgtaacaagaatgtcatcgtcagcatgcttctcctccaccaattgtgcctcctgagctacttct

SEQ ID NO: 29 - DNA, 201 bp, Brassica oleracea
  source 1..201: mol_type = genomic DNA
ttattagatatgaggaaggagaatgtcaaacctagcataagaggaattctggagagatagtagaaccagattaagcttacaaaatatttgtatggttaataagttttgaagttcaaaatcacaattcagtggtcactacaagaaaacacatggttaacgacgaaaattaacgaggaaaaacaatcctcgtaaatttgcgtc

SEQ ID NO: 30 - DNA, 201 bp, Brassica oleracea
  source 1..201: mol_type = genomic DNA
ttattagatatgaggaaggagaatgtcaaacctagcataagaggaattctggagagatagtagaaccagattaagcttacaaaatatttgtatggttaatgagttttgaagttcaaaatcacaattcagtggtcactacaagaaaacacatggttaacgacgaaaattaacgaggaaaaacaatcctcgtaaatttgcgtc

SEQ ID NO: 31 - DNA, 201 bp, Brassica oleracea
  source 1..201: mol_type = genomic DNA
tccatgcttccttcatcagttcctgggtaaacaaggattttaccatggccagcaattcccggtccagctctaaccgcaatcggtttaccccaaccaaaatcgtttccaaacacattgaaccgtggagagctagcaacaacgagcgaattattcaccatcaaaccattcggtattttagggttcttcacccaattttcagca

SEQ ID NO: 32 - DNA, 201 bp, Brassica oleracea
  source 1..201: mol_type = genomic DNA
tccatgcttccttcatcagttcctgggtaaacaaggattttaccatggccagcaattcccggtccagctctaaccgcaatcggtttaccccaaccaaaattgtttccaaacacattgaaccgtggagagctagcaacaacgagcgaattattcaccatcaaaccattcggtattttagggttcttcacccaattttcagca

SEQ ID NO: 33 - DNA, 121 bp, Brassica oleracea
  source 1..121: mol_type = genomic DNA
ggcaacttgtggcgctgtacttcttgtctctttggtttcgtctttgggaatggcctttttatcaaaggcagctaaagcttgctgaaaacataacattctctatctgttagactttcattta

SEQ ID NO: 34 - DNA, 121 bp, Brassica oleracea
  source 1..121: mol_type = genomic DNA
ggcaacttgtggcgctgtacttcttgtctctttggtttcgtctttgggaatggcctttttgtcaatggcagctaaagcttgctgaaaacataacattctctatctgttagactttcattta